(19) United States
(12) Patent Application Publication
(10) Pub. No.: US 2021/0161981 A1
(43) Pub. Date: Jun. 3, 2021

(54) Methods for Liberating Phosphorus from Organic Matter

(71) Applicant: Locus IP Company, LLC, Solon, OH (US)

(72) Inventors: Sean FARMER, North Miami Beach, FL (US); Ken ALIBEK, Solon, OH (US); Paul S ZORNER, Encinitas, CA (US); Alibek MOLDAKOZHAYEV, Solon, OH (US)

(21) Appl. No.: 16/965,357

(22) PCT Filed: Aug. 20, 2019
(86) PCT No.: PCT/US2019/047198

§ 371 Date: Jul. 28, 2020

Related U.S. Application Data
(60) Provisional application No. 62/719,760, filed on Aug. 20, 2018.

(51) Int. Cl. A61K 36/064; C05F 17/20; C05F 17/10; C05F 5/00; C05F 11/08; C12P 3/00; A23K 10/16; A23K 50/80; A23L 33/14; A23L 33/00; A01C 21/00

(52) U.S. Cl. CPC A61K 36/064; C05F 17/20; C05F 17/10; C05F 5/002; C05F 11/08; A23V 2002/00; A23K 10/16; A23K 50/80; A23L 33/14; A23L 33/40; A01C 21/00; C12P 3/00

(57) ABSTRACT

The subject invention provides microbe-based compositions comprising biologically pure yeasts, and/or one or more microbial growth by-products, such as enzymes. In certain embodiments, the enzymes are phytases. Methods of using these compositions to liberate phosphates from phytic acid-containing organic matter are also provided.

### CROSS-REFERENCE TO RELATED APPLICATION

This application claims priority to U.S. Provisional Patent Application No. 62/719,760, filed Aug. 20, 2018, which is incorporated by reference herein in its entirety.

### BACKGROUND OF THE INVENTION

In the agriculture, horticulture, forestry, livestock rearing and aquaculture industries, certain common issues hinder the ability to maximize growth and yields while keeping costs low. These include, but are not limited to, infections and infestations caused by bacteria, fungi, and other pests and pathogens; the high costs of feed; the high cost of chemical fertilizers, as well as their environmental and health impacts; and the difficulty of providing crops and livestock with essential nutrients, such as phosphorus, in usable forms. Additionally, there are growing concerns regarding the depletion of non-renewable resources and the effects of greenhouse gas (GHG) emissions on the health of the environment and the world's ecosystems.

Phosphorus (P) is an essential macronutrient for all living organisms. In plants, P is taken up from the rhizosphere by roots mainly as inorganic phosphate (Pi). Pi is required in large quantities to maximize crop yields. In soil, however, the greatest percentage of phosphorus exists in the form of the phosphorus storage molecule, phytic acid, or phytate in the salt form. Hydrolysis of phytic acid may be carried out by partial acid or basic hydrolysis, or by hydrolysis using phosphatase enzymes, with byproducts including phosphate, inositol and various inositol phosphate intermediates.

The enzymes that catalyze the conversion of phytic acid are known as phytases. Phytases are produced by certain microorganisms, such as, for example, bacteria including Bacillus subtilis and Pseudomonas spp.; yeasts including Saccharomyces cerevisiae; and fungi including Aspergillus terreus and Aspergillus ficuum. Phytases are also endogenously present in small amounts in some plant species.

Phytic acid in soil cannot be taken up by most plants, causing it to accumulate in the soil unused, as well as in the plant detritus and other organic matter in the soil. This may be due to low enzyme levels in the rhizosphere, and/or due to the strong binding of phytate to the soil solid phase. One strategy for overcoming this hurdle involves genetically engineering plant crops to improve plants' capacity to produce exudates and/or enzymes for solubilizing soil-bound phytic acid. However, this process can be costly and complex, and many consumers are opposed to sale and/or consumption of genetically modified crops.

Phosphorus deficiency is also a problem in livestock rearing, and can lead to, for example, infertility, decreased milk production in mammals, and inadequate bone mineralization. Phytic acid is found in many cereals, grains and legumes, but is considered to be an anti-nutritional factor for many non-ruminant animals, which either cannot metabolize the compound, or do so poorly.

Humans and other monogastric animals (e.g., pigs, poultry and fish) produce inadequate quantities, if any, of the enzymes necessary to metabolize phytic acid. Though some hydrolysis of phytic acid does occur in the colon, the inorganic phosphorus has no nutritional value as the inorganic phosphorus is absorbed only in the small intestine. As a consequence, a significant amount of the nutritionally important phosphorus is not used by humans and/or other monogastric animals.

In addition, phytic acid forms complexes with proteins and divalent cations, such as calcium, iron, zinc, magnesium, manganese, copper and molybdenum, thus decreasing bioavailability to an animal's digestive system. For example, ingestion of large quantities of phytic acid containing foods can lead to mineral deficiencies in humans, such as calcium, iron and/or zinc deficiencies. Phytic acid also binds to starch and influences its digestibility and solubility. Thus, monogastric animals must often be given dietary supplements containing inorganic phosphorus to ensure proper growth and health.

Genetic modification of animals and microorganisms has also been attempted to improve the digestion of organic phosphorus compounds. For example, both pigs and E. coli have been engineered to possess genes that code for the production and/or increased production of phytase.

Ruminant animals, on the other hand, can possess microorganisms in the rumen that produce enzymes capable of converting phytic acid into digestible phosphorus. However, for ruminant animals that are free-range and/or are grazing in areas where soils are low in plant-bioavailable phosphorus, dietary supplementation may still be needed in order to combat phosphorus deficiency. Furthermore, when the animals are fed highly-digestible diets (e.g., corn silage), an increased rate of ruminal fluid passage can occur, resulting in incomplete digestion of organic phosphorus compounds.

Human activity over the past century has led to certain environmental concerns related to both phosphorus depletion and phosphorus pollution. Unsustainable farming practices, such as overuse of fertilizers, have steadily depleted the world's recoverable phosphate rock, caused accumulation of unused phosphorus stores in soil, and produced phosphorus runoff into aquifers. Furthermore, runoff from undigested phosphorus stores in livestock manure leaching into groundwater can also be transported into rivers and lakes, leading to eutrophication.

The economic costs of producing food commodities on a large scale, and the depletion of usable phosphorus resources, continue to globally burden human and animal health, and the sustainability of farming and animal husbandry. Genetic modification of plants, pigs and microbes, and dietary supplements for animals and humans are some of the current methods of combatting this issue, but further efforts must be made if a total depletion of phosphorus is to be avoided. Thus, there is a need for methods of sustainable phosphorus management, wherein usable phosphorus can be recovered from organic matter, such as crop waste, in order to recycle and conserve nonrenewable phosphorus resources.

### BRIEF SUMMARY OF THE INVENTION

The subject invention provides microbes, as well as by-products of their growth, such as biosurfactants, metabolites and/or enzymes. The subject invention also provides methods of producing and using these microbes and their by-products. Advantageously, the microbe-based products and methods of the subject invention are environmentally-friendly, operational-friendly and cost-effective.

In preferred embodiments, the subject invention provides microbe-based compositions comprising cultivated microorganisms and/or growth by-products thereof, methods for producing these compositions, and methods of their use in, for example, human health, agriculture, forestry and animal husbandry. Generally, the microbe-based compositions are capable of liberating phosphorus from phytic acid-containing organic matter, such as, for example, soil, plant matter, crop residue and manure. Additionally, the microbe-based compositions can be used for enhancing reforestation and crop growth and yields. Furthermore, the compositions can be used as a dietary supplement for animals and humans, as well as a growth medium supplement for cultivation of microorganisms.

The microorganisms of the subject microbe-based composition are preferably biologically pure yeasts capable of producing one or more useful growth by-products, such as, for example, an enzyme. Preferably, the enzyme is phytase. In specific embodiments, the yeast is Wickerhamomyces anomalus (Pichia anomala) and/or a closely related species (e.g., belonging to the same family and/or genus).

The microbe-based compositions of the subject invention can be obtained through cultivation processes ranging from small to large scale. These cultivation processes include, but are not limited to, submerged cultivation, solid state fermentation (SSF), and hybrids, modifications and/or combinations thereof.

The yeasts in the composition may be in an active or inactive form. Furthermore, the composition can also comprise liquid fermentation broth resulting from cultivation of the yeasts, which can include, inter alia, cellular components and microbial growth by-products, such as biosurfactants, metabolites and/or enzymes. In some embodiments, the composition comprises the fermentation broth without the yeast cells.

Advantageously, direct usage of the composition, i.e., without further stabilization, preservation, and storage, preserves a high viability of the microorganisms, reduces the possibility of contamination from foreign agents and undesirable microorganisms, and/or maintains the activity of the by-products of microbial growth.

In certain embodiments, the composition further comprises one or more biosurfactants. The biosurfactants can be added in purified form, or crude form, where crude form means they are present in a supernatant resulting from cultivation of a biosurfactant-producing microorganism. The crude form can optionally comprise residual nutrients, other microbial growth by-products, microorganisms and/or cellular components. In some embodiments, the biosurfactants are produced by the microorganism(s) of the microbe-based composition.

In some embodiments, the biosurfactants are selected from, for example, glycolipids (e.g., sophorolipids, rhamnolipids, cellobiose lipids, mannosylerythritol lipids and trehalose lipids), lipopeptides (e.g., surfactin, iturin, fengycin, arthrofactin and lichenysin), flavolipids, phospholipids (e.g., cardiolipins), fatty acid ester compounds, and high molecular weight polymers such as lipoproteins, lipopolysaccharide-protein complexes, and polysaccharide-protein-fatty acid complexes.

In certain embodiments, the composition further comprises a carrier. The carrier may be any suitable carrier known in the art that permits the yeasts or yeast by-products to be delivered to target sites in a manner such that the product remains viable, or, in the case of inactive yeast, retains the activity of the components necessary to be effective.

In some embodiments, the composition further comprises a source of organic and/or inorganic phosphorous. For example, flax seeds and almonds are common sources of phytic acid.

The microbe-based composition can be formulated as, for example, a liquid suspension, an emulsion, a freeze- or spray-dried powder, pellets, granules, gels, tablets, capsules, and/or other forms, depending on mode of application and the target site. In certain embodiments, the composition is utilized in liquid form with little to no processing after harvesting from the vessel in which it was cultivated.

In certain embodiments, the compositions of the subject invention have advantages over, for example, purified microbial metabolites alone, due to, for example, the use of the entire microbial culture. These advantages include one or more of the following: high concentrations of mannoprotein as a part of a yeast cell wall's outer surface; the presence of beta-glucan in yeast cell walls; the presence of biosurfactants in the culture; and the presence of solvents and other metabolites (e.g., vitamins, minerals, carbohydrates and protein sources) in the culture. These advantages are present when using active or inactive yeast.

In certain embodiments, the microbes, as well as the metabolites and other by-products of the microbes, work synergistically with one another.

In one embodiment, the subject invention provides methods of producing a growth by-product of a microorganism, wherein a microorganism is cultivated under conditions appropriate for growth and production of the growth by-products; and, optionally, purifying the by-products. In some embodiments, a source of phytic acid is included in the growth medium, such as plant detritus.

In some embodiments, the produced growth by-product is not purified, but instead utilized in a crude form, e.g., comprising the fermentation medium in which it was produced. Examples of growth by-products according to the subject invention include enzymes, acids, solvents, ethanol, proteins, amino acids, biosurfactants, and others. In specific embodiments, methods are provided for producing a microbe-based composition comprising the enzyme phytase.

The subject invention further provides methods of liberating phosphorus, e.g. in the form of inorganic phosphates, from phytic acid present in organic matter, wherein the methods comprise applying an effective amount of a microbe-based composition of the subject invention to the organic matter. The microbes can be either live (viable) or inactive at the time of application.

In the case of live microorganisms, the microorganisms can grow in situ at the site of application and produce active compounds or growth by-products onsite. Consequently, a high concentration of microorganisms and beneficial growth by-products can be achieved easily and continuously at a treatment site.

To this end, the methods can comprise adding materials to enhance microbial growth during application (e.g., adding nutrients and/or prebiotics to promote microbial growth).

In one embodiment, the methods further comprise a step of cultivating the microbe-based composition prior to application. Preferably, all or part of the microbe-based composition is cultivated at or near the site of application, for example, less than 300 miles from the site.

Advantageously, when the composition is contacted with the organic matter according to the subject methods, the microbial-produced phytase in the composition can react with the phytic acid in the organic matter, catalyzing hydrolysis of the phytic acid and causing a release of usable phosphorus byproducts, e.g., in the form of inorganic phosphates, over time.

In certain embodiments, the organic matter is organic waste matter, such as post-harvest crop residue, which can include, for example, leftover corn stalks, corn stover, corn cobs, wheat straw, soybean straw, rice hulls, and other plant stems, leaves, roots and parts.

Other types of organic matter are also envisioned, including, for example, plant-based matter such as nuts, seeds and legumes, plant-based compost, manure, leftovers from corn, cellulosic or biomass ethanol production (e.g., distiller's grains, lignin and brewers' spent grain), saw dust, used coffee grounds, and yard waste (e.g., tree, hedge and lawn clippings).

In some embodiments, the speed at which phosphate release occurs can be enhanced by chopping, crushing or otherwise reducing the size of any individual pieces of the organic matter prior to applying the microbe-based composition.

In one embodiment, the composition is poured, sprayed or sprinkled onto the organic matter and then, optionally, mixed using any standard mixing device or technique known in the art. Further components can also be applied, such as, for example, water or nutrients (e.g., nutrients for microbial growth and/or for plant growth).

In one embodiment, the organic waste matter is crop residue that is leftover on a post-harvest crop field. As crop residue decomposes, nutrients that are necessary for plant growth are released into the soil. The subject invention can be used to convert unavailable forms of phosphorus that are released by this decomposition process into plant-absorbable forms. For example, the composition can be applied directly onto crop residue that is left behind on a field. The crop residue and composition can be left on the surface of the soil, or they can be tilled into the soil.

In one embodiment, the composition is added to compost prior to applying the compost to a field, allowing the phytase in the composition to catalyze the hydrolysis of phytic acid, and then applying the compost to a field or forest as fertilizer.

The composition can also be applied to organic matter that has been collected from its source and mixed together at another location. The treated organic matter can then be transported to a desired application site, such as, for example, a crop field and used as, for example, a biofertilizer.

In one embodiment of the subject methods, the organic matter is soil. By applying the subject composition directly onto the soil and either mixing the composition into the soil or allowing it to percolate into the soil, the method can be used to enrich the soil by replenishing it with plant-absorbable phosphorus stores. Advantageously, the methods can increase yields and enhance the quality of crops and produce due to the liberation of phosphates from phytate or phytic acid in the soil.

In one embodiment, methods of enhancing production in animal husbandry (e.g., livestock rearing or aquaculture) are provided, wherein the microbe-based composition is applied to an animal's water and/or feed as a dietary supplement and/or digestive aide. In one embodiment, methods of enhancing human health are provided, wherein the microbe-based composition is administered to a human as a dietary supplement and/or digestive aide. Advantageously, the presence of phytase in the composition allows for increased absorption of phosphorus from food sources that may naturally contain phytic acid and/or phosphates, reduces the amount of inorganic phosphate needed to supplement an animal's feed, and helps prevent phosphorus deficiency.

In certain embodiments, due to the presence of advantageous biochemical-producing microorganisms in the subject microbe-based compositions, the subject methods can also help with preventing harmful organisms from harboring in organic waste matter. For example, manure and decomposing crop residue can be attractive for certain pests, fungi and bacteria that might be harmful to plants that are grown with the manure or residue. Killer yeasts, such as Wickerhamomyces anomalus, are capable of producing metabolites that are useful for controlling many of these unwanted pests.

The methods of the subject invention allow for the recycling of organic waste material, as well as the release of vitamins, minerals and importantly, phosphorus, that remain in certain organic matter. Furthermore, the compositions and methods utilize components that are biodegradable and toxicologically safe. Thus, the present invention can be used for enhancing production in agriculture, forestry, and animal husbandry, as well as enhancing human health, as a “green” treatment.

### DETAILED DESCRIPTION OF THE INVENTION

In preferred embodiments, the subject invention provides microbe-based compositions comprising cultivated microorganisms and/or microbial growth by-products, methods for producing these compositions, and methods of their use in, for example, human health, agriculture, forestry, and animal husbandry. Generally, the microbe-based compositions are capable of liberating phosphorus from phytic acid-containing organic matter, such as, for example, soil, plant matter, crop residue and manure. Additionally, the microbe-based compositions can be used for enhancing reforestation and crop growth and yields. Furthermore, the compositions can be used as a dietary supplement for animals and humans, to, for example, prevent phosphorus deficiency, as well as a growth medium supplement for cultivation of microorganisms.

The microorganism of the subject microbe-based composition is preferably a biologically pure yeast capable of producing one or more useful growth by-products, such as, for example, an enzyme. Preferably, the enzyme is phytase. In a specific embodiment, the yeast is, Wickerhamomyces anomalus (Pichia anomala), or a yeast closely related thereto (e.g., belonging to the same genus and/or family).

The subject invention further provides methods of liberating phosphates from phytic acid present in organic matter, wherein the methods comprise applying an effective amount of a microbe-based composition of the subject invention to the organic matter. The microbes can be either live (viable) or inactive at the time of application. Advantageously, when the composition is contacted with the organic matter according to the subject methods, the microbial-produced phytase in the composition can catalyze the hydrolysis of the phytic acid in the organic matter, causing a release of usable phosphorus byproducts, e.g., in the form of inorganic phosphates, over time.

### Selected Definitions

As used herein, reference to a “microbe-based composition” means a composition that comprises components that were produced as the result of the growth of microorganisms or other cell cultures. Thus, the microbe-based composition may comprise the microbes themselves and/or by-products of microbial growth. The microbes may be planktonic or in a biofilm form, or a mixture of both. The by-products of growth may be, for example, metabolites (e.g., biosurfactants, enzymes), cell membrane components, proteins, and/or other cellular components. The microbes may be intact or lysed. The cells may be absent from the composition, or present at a concentration of, for example, at least 1×10⁴, 1×10⁵, 1×10⁶, 1×10⁷, 1×10⁸, 1×10⁹, 1×10¹⁰, 1×10¹¹, 1×10¹² or more CFU per milliliter of the composition.

The subject invention further provides “microbe-based products,” which are products that are to be applied in practice to achieve a desired result. The microbe-based product can be simply the microbe-based composition harvested from the microbe cultivation process. Alternatively, the microbe-based product may comprise further ingredients that have been added. These additional ingredients can include, for example, stabilizers, buffers, carriers (e.g., water or salt solutions), added nutrients to support further microbial growth, non-nutrient growth enhancers and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, “harvested” in the context of cultivation of a microorganism refers to removing some or all of a microbe-based composition from a growth vessel.

As used herein, an “isolated” or “purified” molecule or compound is substantially free of other compounds, such as cellular material, with which it is associated in nature. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state.

As used here in, a “biologically pure culture” is one that has been isolated from materials with which it is associated in nature. In a preferred embodiment, the culture has been isolated from all other living cells. In further preferred embodiments, the biologically pure culture has advantageous characteristics compared to a culture of the same microbe as it exists in nature. The advantageous characteristics can be, for example, enhanced production of one or more desirable growth by-products.

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism (i.e., a growth by-product) or a substance necessary for taking part in a particular metabolic process. A metabolite can be an organic compound that is a starting material, an intermediate in, or an end product of metabolism. Examples of metabolites include, but are not limited to, biopolymers, enzymes, acids, solvents, alcohols, proteins, vitamins, minerals, microelements, amino acids, and biosurfactants.

As used herein, “modulate” is interchangeable with alter (e.g., increase or decrease). Such alterations are detected by standard art known methods such as those described herein.

Ranges provided herein are understood to be shorthand for all of the values within the range. For example, a range of 1 to 20 is understood to include any number, combination of numbers, or sub-range from the group consisting of 1, 2, 3, 4, 5, 6, 7, 8, 9, 10, 11, 12, 13, 14, 15, 16, 17, 18, 19, 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, “reduces” refers to a negative alteration of at least 1%, 5%, 10%, 25%, 50%, 75%, or 100%, and “increases” refers to a positive alteration of at least 1%, 5%, 10%, 25%, 50%, 75%, or 100%.

As used herein, “reference” refers to a standard or control condition.

As used herein, “surfactant” refers to a surface-active agent that lowers the surface tension (or interfacial tension) between a liquid and a gas, between two liquids or between a liquid and a solid. Surfactants act as, e.g., detergents, wetting agents, emulsifiers, foaming agents, and dispersants. A “biosurfactant” is a surfactant produced by a living organism.

As used herein, reference to “phytic acid” includes the terms inositol hexakisphosphate, IP6, inositol polyphosphate, phytate and phytin, and any other salts and/or forms of the phytic acid molecule.

As used herein, “agriculture” means the cultivation and breeding of plants and/or fungi for food, fiber, biofuel, medicines, cosmetics, supplements, ornamental purposes and other uses. According to the subject invention, agriculture can also include horticulture, landscaping, gardening, plant conservation, orcharding and arboriculture. Further included in agriculture herein is soil science (e.g., pedology and edaphology) as well as agronomy, or the care, monitoring and management of soil and crop production.

As used herein, “livestock” refers to any domesticated animal raised in an agricultural or industrial setting to produce commodities such as food, fiber and labor. “Livestock rearing” is considered a form of animal husbandry, and includes the breeding, raising, rearing, maintenance and/or slaughter of these animals. Livestock can be produced free-range, such as on open fields, on farms, or in animal feeding operations. Types of animals included in the term livestock can include, but are not limited to, alpacas, beef and dairy cattle, bison, pigs, sheep, goats, horses, mules, asses, dogs, camels, chickens, turkeys, ducks, geese, guinea fowl, and squabs.

As used herein, “aquaculture,” “aquafarming,” “aquatic farming,” “aquatic husbandry” or “fish farming” is a form of animal husbandry, and includes the breeding, rearing, and harvesting of aquatic animals in a fish farm. Aquaculture can be intensive (relying on technology to raise fish in artificial enclosures at high densities) or extensive (performed in the ocean, or in natural and man-made lakes, bays, rivers, fjords, or other bodies of water). Aquaculture includes the production of seafood from hatchery fish and shellfish which are grown to market size in enclosures, ponds, tanks, aquariums, cages, or raceways. Additionally, aquaculture includes mariculture, which entails the culture of marine organisms in open seawater or enclosed sections of seawater. Furthermore, aquaculture includes stock restoration or enhancement, wherein hatchery fish and shellfish are released into the wild in an effort to rebuild wild populations or coastal habitats. Even further, aquaculture includes the production of ornamental fish for the aquarium trade, as well as the husbandry of ornamental fish housed within aquariums. Species that can be farmed include freshwater or saltwater fish and shellfish, and can include ornamental fish, food fish, sport fish, bait fish, crustaceans, mollusks, algae, sea vegetables, or fish eggs.

As used herein, a “fish farm” is any water environment wherein aquaculture occurs or can occur. Fish farms according to this disclosure can include all types of water environments or sections of water environments, whether man-made or naturally occurring, including ponds, irrigation ditches, rivers, lakes, oceans, fjords, tanks, aquariums, cages, or raceways.

As used herein, a “pest” is any organism, other than a human, that is destructive, deleterious and/or detrimental to humans or human concerns (e.g., agriculture, horticulture, animal husbandry). Pests may cause infections, infestations and/or disease. Pests may be single- or multi-cellular organisms, including but not limited to, arthropods, viruses, fungi, bacteria, parasites, protozoa, and/or nematodes.

As used herein, “treating” refers to eradicating, reducing, ameliorating, reversing, or preventing a degree, sign or symptom of a condition or disorder, and includes, but does not require, a complete cure of the condition or disorder. Treating can be curing, improving, or partially ameliorating a disorder.

As used herein, “preventing” a situation or occurrence, means delaying the onset or progression thereof. In some instances, prevention may not be absolute, meaning that the situation or occurrence still may occur, but with delay.

### Microbe-Based Compositions

The subject invention provides microbe-based compositions comprising beneficial microorganisms, as well as one or more microbial growth by-products, such as biosurfactants, metabolites, acids, solvents and/or enzymes. Furthermore, the subject invention provides materials and methods for producing the microbe-based compositions.

Advantageously, the microbe-based compositions according to the subject invention are non-toxic (e.g., ingestion toxicity is greater than 5 g/kg of body weight) and can be applied in high concentrations without causing irritation and/or toxicity to, for example, a human or animal's skin or digestive tract.

In certain embodiments, the microbes of the subject invention are biologically pure killer yeasts. In particular, the subject invention utilizes killer yeasts belonging to the genus Pichia. Even more specifically, in one embodiment, the microbes are Wickerhamomyces anomalus (Pichia anomala). Other phytase-producing yeasts, such as Pichia kudriavzevii, P. guilliermondii, and P. occidentalis may also be utilized.

In one embodiment, the composition comprises about 1×10⁶ to 1×10¹², 1×10⁷ to 1×10¹¹, 1×10⁸ to 1×10¹⁰, or 1×10⁹ CFU/ml of the microorganism(s).

In certain embodiments, the microbe-based composition of the subject invention can comprise fermentation broth containing a live and/or an inactive culture and/or the microbial metabolites produced by the microorganism and/or any residual nutrients. The composition may be, for example, at least, by weight, 1%, 5%, 10%, 25%, 50%, 75%, or 100% broth. The amount of biomass in the composition, by weight, may be, for example, anywhere from 0% to 100% inclusive of all percentages therebetween, or, for example from 5 g/l to 180 g/l or more, or from 10 g/l to 150 g/l.

The product of fermentation may be used directly without extraction or purification. If desired, extraction and purification can be easily achieved using standard extraction and/or purification methods or techniques described in the literature.

In some embodiments, the composition further comprises additional crude form or purified microbial growth-products, such as enzymes, biosurfactants, solvents, acids, proteins, minerals and/or vitamins. Crude form metabolites can take the form of, for example, a liquid mixture comprising metabolite sediment in fermentation broth resulting from cultivation of a microbe. This crude form solution can comprise from about 25% to about 75%, from about 30% to about 70%, from about 35% to about 65%, from about 40% to about 60%, from about 45% to about 55%, or about 50% pure metabolite.

In preferred embodiments, the composition comprises a phosphatase enzyme, such as phytase. Phytase catalyzes the hydrolysis of phytic acid or phytate, which is an organic form of phosphorus that releases a form of inorganic phosphorus upon hydrolysis. The phytase can be present in the composition as the result of growth of the microbes present in the composition, or the phytase can be produced separately by other phytase-producing microorganisms and added to the composition in crude form and/or purified form.

In certain embodiments, the concentration of phytase (or other enzyme) in the composition is from 1 to 10,000 u/ml, from 100 to 9,000 u/ml, or from 200 to 8,000 u/ml.

Additionally, in one embodiment, the composition comprises biosurfactants. The biosurfactants can be present in the composition as the result of growth of the microbes present in the composition, or the biosurfactants can be produced separately by other microorganisms and added to the composition in crude form and/or purified form.

Biosurfactants inhibit microbial adhesion to a variety of surfaces, prevent the formation of biofilms, and can have powerful emulsifying and demulsifying properties. Additionally, biosurfactants are capable of reducing surface and interfacial tension of water in, for example, fish farms and aquariums.

In some embodiments, the biosurfactants are selected from, for example, glycolipids (e.g., sophorolipids, rhamnolipids, cellobiose lipids, mannosylerythritol lipids and trehalose lipids), lipopeptides (e.g., surfactin, iturin, fengycin, arthrofactin and lichenysin), flavolipids, phospholipids (e.g., cardiolipins), fatty acid ester compounds, and high molecular weight polymers such as lipoproteins, lipopolysaccharide-protein complexes, and polysaccharide-protein-fatty acid complexes.

In certain embodiments, the concentration of the one or more biosurfactants in the composition is 0.001 to 90 by weight % (wt %), preferably 0.01 to 50 wt %, and more preferably 0.1 to 20 wt %. The biosurfactants can further be present at about 0.01 g/L to about 500 g/L, about 0.5 g/L to about 50.0 g/L, from about 1.0 to about 10.0 g/L or from about 2.0 to about 5.0 g/L.

### Soil Enrichment Formulation

In one embodiment, the composition is preferably formulated for application to soil, seeds, whole plants, or plant parts (including, but not limited to, roots, tubers, sterns, flowers and leaves). In certain embodiments, the composition is formulated as, for example, liquid, dust, granules, microgranules, pellets, wettable powder, flowable powder, emulsions, microcapsules, oils, or aerosols.

To improve or stabilize the effects of the composition, it can be blended with suitable adjuvants and then used as such or after dilution, if necessary. In preferred embodiments, the composition is formulated as a liquid, a concentrated liquid, or as dry powder or granules that can be mixed with water and other components to form a liquid product.

In one embodiment, the composition can comprise glucose (e.g., in the form of molasses), glycerol and/or glycerin, as, or in addition to, an osmoticum substance, to promote osmotic pressure during storage and transport of the dry product.

The compositions can be used either alone or in combination with other compounds and/or methods for efficiently enhancing plant health, growth and/or yields, and/or for supplementing the growth of the first and second microbes. For example, in one embodiment, the composition can include and/or can be applied concurrently with nutrients and/or micronutrients for enhancing plant and/or microbe growth, such as magnesium, phosphate, nitrogen, potassium, selenium, calcium, sulfur, iron, copper, and zinc; and/or one or more prebiotics, such as kelp extract, fulvic acid, chitin, humate and/or humic acid. The exact materials and the quantities thereof can be determined by a grower or an agricultural scientist having the benefit of the subject disclosure.

The compositions can also be used in combination with other agricultural compounds and/or crop management systems. In one embodiment, the composition can optionally comprise, or be applied with, for example, natural and/or chemical pesticides, repellants, herbicides, fertilizers, water treatments, non-ionic surfactants and/or soil amendments. Preferably, however, the composition does not comprise and/or is not used with benomyl, dodecyl dimethyl ammonium chloride, hydrogen dioxide/peroxyacetic acid, imazilil, propiconazole, tebuconazole, or triflumizole.

If the composition is mixed with compatible chemical additives, the chemicals are preferably diluted with water prior to addition of the subject composition.

Further components can be added to the composition, for example, buffering agents, carriers, other microbe-based compositions produced at the same or different facility, viscosity modifiers, preservatives, nutrients for microbe growth, tracking agents, biocides, other microbes, surfactants, emulsifying agents, lubricants, solubility controlling agents, pH adjusting agents, preservatives, stabilizers and ultra-violet light resistant agents.

### Human or Animal Dietary Supplement Formulation

In certain embodiments, the composition is formulated as a human and/or animal dietary supplement or digestive aide.

In certain embodiments, the use of the yeast in the feed provides rich sources of protein and/or polysaccharides. In one embodiment, the subject composition can comprise additional nutrients to supplement a human and/or animal's diet and/or promote health and/or well-being, such as, for example, sources of amino acids (including essential amino acids), peptides, proteins, vitamins, microelements, fats, fatty acids, lipids, carbohydrates, sterols, enzymes, prebiotics, and trace minerals.

Preferred compositions comprise vitamins and/or minerals in any combination. Vitamins for use in a composition of this invention can include for example, vitamins A, E, K3, D3, B1, B3, B6, B12, C, biotin, folic acid, panthothenic acid, nicotinic acid, choline chloride, inositol and para-amino-benzoic acid. Minerals can include, for example, salts of calcium, cobalt, copper, iron, magnesium, potassium, selenium and zinc. Other components may include, but are not limited to, antioxidants, beta-glucans, bile salt, cholesterol, enzymes, carotenoids, and many others. Typical vitamins and minerals are those, for example, recommended for daily consumption and in the recommended daily amount (RDA), although precise amounts can vary. The composition would preferably include a complex of the RDA vitamins, minerals and trace minerals as well as those nutrients that have no established RDA, but have a beneficial role in healthy mammal physiology.

In some embodiments, sources of phytate and/or phytic acid are pre-mixed with the composition, so that when contacted with the phytase in the composition, the composition can provide readily available inorganic phosphorous upon administration to a human or animal subject. Sources of phytate and/or phytic acid can be, for example, almonds, walnuts, flax seeds, legumes, oats, and whole grains.

In one embodiment, the composition can further comprise pre-made wet or dry animal feed, wherein the pre-made food has been cooked and/or processed to be ready for animal consumption. For example, the microorganism and/or growth by-products can be poured onto and/or mixed with the pre-made food, or the microorganism and/or growth by-products can serve as a coating on the outside of dry animal food pieces, e.g., morsels, kibbles or pellets.

In one embodiment, the composition can further comprise raw ingredients for making animal feed, wherein the raw ingredients, together with the microorganism and/or growth by-products, are then cooked and/or processed to make an enhanced dry or wet feed product.

In some embodiments, the composition further comprises prebiotics to support growth of beneficial microbes in the gut. Prebiotics can include, for example, fermentable fibers derived from fructans and xylans, inulin, fructooligosaccharides, xylooligosaccahrides and galactooligosaccharides. Foods known to contain prebiotics include, for example, chicory root, onions, garlic, leek, oatmeal, wheat bran, asparagus, dandelion greens, Jerusalem artichoke, and banana.

In one embodiment, the composition can be formulated as an orally-consumable product and administered orally to an animal or human subject.

Orally-consumable products according to the invention are any preparations or compositions suitable for consumption, for nutrition, for oral hygiene or for pleasure, and are products intended to be introduced into the human or animal oral cavity, to remain there for a certain period of time and then to either be swallowed (e.g., supplements, food ready for consumption) or to be removed from the oral cavity again (e.g. chewing gums or products of oral hygiene or medical mouth washes). These products include all substances or products intended to be ingested by humans or animals in a processed, semi-processed or unprocessed state. This also includes substances that are added to orally consumable products (particularly food and pharmaceutical products) during their production, treatment or processing and intended to be introduced into the human or animal oral cavity.

Orally-consumable products can also include substances intended to be swallowed by humans or animals and then digested in an unmodified, prepared or processed state; the orally consumable products according to the invention therefore also include casings, coatings or other encapsulations that are intended also to be swallowed together with the product or for which swallowing is to be anticipated.

In certain embodiments, the composition can further comprise components that are, for example, sources of energy, nutrients and/or other health-promoting supplements, flavorings, preservatives, pH adjusters, sweeteners and/or dyes.

In one embodiment, the composition is formulated as a dehydrated powder or concentrate that can be reconstituted into a drinkable fluid by the addition of water. In one embodiment, the composition is formulated as a blended smoothie or milkshake.

In one embodiment, the composition can be formulated for administering directly into the GI tract. For example, the composition can be formulated for administration to the proximal lower GI via colonoscopy, the distal lower GI via enema or rectal tubes, and the upper GI tract via nasogastric tubes, duodenal tubes, and endoscopy/gastroscopy.

In certain embodiments, the present invention can be used to enhance a human or animal's overall health and well-being by increasing phosphorous absorption in the human or animal's digestive tract.

### Fermentation of Microorganisms

The subject invention utilizes methods for cultivating microorganisms and producing microbial metabolites and/or growth by-products. More specifically, the subject invention provides materials and methods for the production of biomass, extracellular metabolites, residual nutrients and/or intracellular components.

The subject invention utilizes cultivation processes that are suitable for cultivation of microorganisms and production of microbial metabolites on any desired scale, from small (e.g., lab setting) to large (e.g., industrial setting). These cultivation processes include, but are not limited to, submerged cultivation/fermentation, solid state fermentation (SSF), and combinations, hybrids and/or modifications thereof.

As used herein “fermentation” or “cultivation” refers to growth of cells under controlled conditions. The growth could be aerobic or anaerobic.

The microbe growth vessel used according to the subject invention can be any fermenter or cultivation reactor for industrial use. In one embodiment, the vessel may have functional controls/sensors or may be connected to functional controls/sensors to measure important factors in the cultivation process, such as pH, oxygen, pressure, temperature, agitator shaft power, humidity, viscosity and/or microbial density and/or metabolite concentration.

The vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique.

In preferred embodiments, a microbe growth facility comprising multiple microbe growth vessels produces fresh, high-density microorganisms and/or microbial growth by-products of interest on a desired scale. The microbe growth facility may be located at or near the site of application. The facility produces high-density microbe-based compositions in batch, quasi-continuous, or continuous cultivation.

The distributed microbe growth facilities can be located at the location where the microbe-based product will be used (e.g., a field or fish farm). For example, the microbe growth facility may be less than 300, 250, 200, 150, 100, 75, 50, 25, 15, 10, 5, 3, or 1 mile from the location of use, or can be located directly on the site of use.

In certain embodiments, production may or may not be achieved using local and/or distributed fermentation methods, meaning that conventional methods can also be utilized according to the subject invention. However, local and/or distributed microbe growth facilities as described herein advantageously provide a solution to the current problem of relying on far-flung industrial-sized producers whose product quality suffers due to upstream processing delays, supply chain bottlenecks, improper storage, and other contingencies that inhibit the timely delivery and application of a useful product.

The microbe growth facilities produce fresh, microbe-based compositions, comprising the microbes themselves, microbial metabolites, and/or other components of the broth in which the microbes are grown. If desired, the compositions can have a high density of vegetative cells, inactive cells, propagules, or a mixture of vegetative cells, inactive cells and/or propagules.

Advantageously, the compositions can be tailored for use at a specified location. In one embodiment, the microbe growth facility is located on, or near, a site where the microbe-based products will be used. The microbe growth facilities may operate off the grid by utilizing, for example, solar, wind, and/or hydroelectric power.

The microbe growth facilities provide manufacturing versatility by the ability to tailor the microbe-based products to improve synergies with destination geographies. For example, the systems of the subject invention are capable of harnessing the power of naturally-occurring local microorganisms and their metabolic by-products. Local microbes can be identified based on, for example, salt tolerance, ability to grow at high temperatures, and/or ability to produce certain metabolites.

Because the microbe-based product is generated on-site or near the site of application, without the requirement of stabilization, preservation, prolonged storage and extensive transportation processes of conventional production, a much higher density of live (or inactive) microorganisms and/or propagules thereof can be generated, thereby requiring a much smaller volume of the microbe-based product for use in an on-site application or allowing for much higher density of microbial applications where necessary. This reduces the possibility of contamination from foreign agents and undesirable microorganisms, maintains the activity of the by-products of microbial growth, and allows for an efficient scaled-down bioreactor (e.g. smaller fermentation tank and smaller volume of starter materials, nutrients, pH control agents, and de-foaming agent, etc.), with no reason to stabilize the cells. Locally-produced high density, robust cultures of microbes are more effective in the field than those that have undergone vegetative cell stabilization or have been sitting in the supply chain for some time.

Local generation of the microbe-based product also facilitates the inclusion of the fermentation broth in the product. The broth can contain agents produced during the fermentation that are particularly well-suited for local use. This further facilitates the portability of the product.

Reduced transportation times allow for the production and delivery of fresh batches of microbes and/or their metabolites at the time and volume as required by local demand. Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

In one embodiment, the method of cultivation, whether performed using conventional methods or using local or distributed systems, utilizes a culture medium comprising molasses, urea and peptone.

In one embodiment, the concentration of molasses is from 2 to 6%, preferably 4%. In one embodiment, the concentration of urea is from 0.01 to 1.0%, preferably 0.2%. In one embodiment, the concentration of peptone is from 1.0 to 5%, preferably 2.5%.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method of cultivation can provide oxygenation to the growing culture. One embodiment utilizes slow motion of air to remove low-oxygen containing air and introduce oxygenated air. The oxygenated air may be ambient air supplemented daily through mechanisms including impellers for mechanical agitation of the liquid, and air spargers for supplying bubbles of gas to the liquid for dissolution of oxygen into the liquid.

The method can further comprise supplementing the cultivation with a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, rice bran oil, canola oil, sunflower oil, olive oil, corn oil, sesame oil, and/or linseed oil. These carbon sources may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included, e.g., L-Alanine.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, calcium carbonate, and/or sodium carbonate. These inorganic salts may be used independently or in a combination of two or more.

In some embodiments, the method for cultivation may further comprise adding additional acids and/or antimicrobials in the liquid medium before, and/or during the cultivation process. Antimicrobial agents or antibiotics can be used for protecting the culture against contamination. For example, Streptomyces erythromycin, hops or hop acid, and/or small amounts, e.g., 50-100 ppm, of sophorolipids or other biosurfactants can be added to nutrient medium as antibacterial agents. Additionally, antifoaming agents may also be added to prevent the formation and/or accumulation of foam during cultivation.

The pH of the mixture should be suitable for the microorganism of interest. Buffers, and pH regulators, such as carbonates and phosphates, may be used to stabilize pH near a preferred value. pH control can also be used for preventing contamination of the culture. For example, cultivation can be initiated at low pH that is suitable for yeast growth (e.g., 3.0-3.5), and then increased after yeast accumulation (e.g., to 4.5-5.0) and stabilized for the remainder of fermentation. When metal ions are present in high concentrations, use of a chelating agent in the liquid medium may be necessary.

The microbes can be grown in planktonic form or as biofilm. In the case of biofilm, the vessel may have within it a substrate upon which the microbes can be grown in a biofilm state. The system may also have, for example, the capacity to apply stimuli (such as shear stress) that encourages and/or improves the biofilm growth characteristics.

In one embodiment, the method for cultivation of microorganisms is carried out at about 5° to about 100° C., preferably, 15 to 60° C., more preferably, 25 to 50° C. In a further embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control bacterial growth.

In other embodiments, the cultivation system may be self-sterilizing, meaning the organism being cultivated is capable of preventing contamination from other organisms due to production of antimicrobial growth by-products or metabolites.

In one embodiment, surfactants, enzymes, metabolites, and/or other proteins are produced by cultivating a microbe strain of the subject invention under conditions appropriate for growth and production thereof; and, optionally, concentrating and purifying the microbial growth by-product of interest. In preferred embodiments, the growth by-product is an enzyme, even more preferably, phytase.

The microbial growth by-product produced by microorganisms of interest may be retained in the microorganisms or secreted into the growth medium. Optionally, the growth medium may contain compounds that stabilize the activity of microbial growth by-product.

The biomass content of the fermentation broth may be, for example, from 5 g/l to 180 g/l or more, or from 10 g/l to 150 g/l.

When it is time to harvest the microbe-based product from the growth vessel or vessels, the microbes and/or broth resulting from the microbial growth can be removed from the growth vessel and transferred via, for example, piping for immediate use. The microorganisms may be in an active or inactive form, or may contain a combination of active and inactive microorganisms.

The composition (microbes, broth, or microbes and broth) can also be placed in containers of appropriate size, taking into consideration, for example, the intended use, the contemplated method of application, the size of the fermentation tank, and any mode of transportation from microbe growth facility to the location of use. Thus, the containers into which the microbe-based composition is placed may be, for example, from 1 gallon to 1,000 gallons or more. In certain embodiments the containers are 2 gallons, 5 gallons, 25 gallons, or larger.

Further components can be added as the harvested product is placed into containers and/or piped (or otherwise transported for use). The additives can be, for example, buffers, carriers, other microbe-based compositions produced at the same or different facility, viscosity modifiers, preservatives, nutrients for microbe growth, tracking agents, pesticides, and other ingredients specific for an intended use.

In one embodiment, all of the microbial cultivation composition is removed upon the completion of the cultivation (e.g., upon, for example, achieving a desired cell density, or density of a specified metabolite in the broth). In this batch procedure, an entirely new batch is initiated upon harvesting of the first batch.

In another embodiment, only a portion of the fermentation product is removed at any one time. In this embodiment, biomass with viable cells remains in the vessel as an inoculant for a new cultivation batch. The composition that is removed can be a cell-free broth or can contain cells. In this manner, a quasi-continuous system is created.

### Preparation of Microbe-based Products

One microbe-based product of the subject invention is simply the fermentation broth containing the microorganism and/or the microbial metabolites produced by the microorganism and/or any residual nutrients. The product of fermentation may be used directly without extraction or purification. If desired, extraction and purification can be easily achieved using standard extraction and/or purification methods or techniques described in the literature.

The microorganisms in the microbe-based product may be in an active or inactive form. The microbe-based products may contain combinations of active and inactive microorganisms.

The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

The microbes and/or broth resulting from the microbial growth can be removed from the growth vessel and transferred via, for example, piping for immediate use. In other embodiments, as described previously, the composition (microbes, broth, or microbes and broth) can be placed in containers of appropriate size.

Upon harvesting the microbe-based composition from the growth vessels, further components can be added as the harvested product is placed into containers and/or piped (or otherwise transported for use). The additives can be, for example, buffering agents, carriers, adjuvants, other microbe-based compositions produced at the same or different facility, viscosity modifiers, preservatives, nutrients for microbe growth, tracking agents, biocides, other microbes, non-biological surfactants, emulsifying agents, lubricants, buffering agents, solubility controlling agents, pH adjusting agents, stabilizers, ultra-violet light resistant agents and other ingredients specific for an intended use.

In one embodiment, the composition may further comprise buffering agents including organic and amino acids or their salts, to stabilize pH near a preferred value. The pH of the microbe-based composition should be suitable for the microorganism of interest.

Suitable buffers include, but are not limited to, citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and mixtures thereof. Phosphoric and phosphorus acids or their salts may also be used. Synthetic buffers are suitable to be used but it is preferable to use natural buffers such as organic and amino acids or their salts.

In a further embodiment, pH adjusting agents include potassium hydroxide, ammonium hydroxide, potassium carbonate or bicarbonate, hydrochloric acid, nitric acid, sulfuric acid and mixtures thereof.

In one embodiment, additional components such as an aqueous preparation of a salt, such as sodium bicarbonate or carbonate, sodium sulfate, sodium phosphate, or sodium biphosphate, can be included in the microbe-based composition.

Advantageously, the microbe-based product may comprise broth in which the microbes were grown. The product may be, for example, at least, by weight, 1%, 5%, 10%, 25%, 50%, 75%, or 100% broth. The amount of biomass in the product, by weight, may be, for example, anywhere from 0% to 100% inclusive of all percentages therebetween.

In certain embodiments, the microbe-based composition of the subject invention further comprises a carrier. The carrier may be any suitable carrier known in the art that permits the yeasts or yeast growth by-products to be delivered to target plants, soil, animals, fish, etc. in a manner such that the product remains viable, or, in the case of inactive yeast, retains the components necessary to be effective.

Carriers can be comprised of solid-based, dry materials for formulation into tablet, capsule, granule or powdered form; or the carrier can be comprised of liquid or gel-based materials for formulations into liquid or gel forms. For example, carriers can comprise water, saline, biopolymers, natural plant fibers, materials such as clay, silage, vermiculite, pumice, or paper sludge.

In certain embodiments, particularly in the context of agriculture, the microbe-based composition can further comprise an adjuvant to increase the efficacy of the composition. In one embodiment, the adjuvant is selected from one or more of kelp extract, chitin, fulvic acid, humic acid and humate.

Optionally, the composition can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C. On the other hand, a biosurfactant composition can typically be stored at ambient temperatures.

In certain embodiments, the microbe-based products of the subject invention have advantages over, for example, purified microbial metabolites alone, due to, for example, the use of the entire microbial culture. These advantages include one or more of the following: high concentrations of mannoprotein as a part of a yeast cell wall's outer surface; the presence of beta-glucan in yeast cell walls; the presence of biosurfactants in the culture; and the presence of solvents and other metabolites in the culture. These advantages are present when using active or inactive yeast.

The microbe-based composition can be formulated as a microbe-based product in the form of, for example, a liquid suspension, emulsion, freeze- or spray-dried powder, granules, pellets, or a gel. Preferably, the composition is utilized in liquid form, although other formulations are envisioned as they are appropriate for a particular application.

In one embodiment, particularly for use in livestock or aquaculture applications, the microbe-based product can be formulated as a feed pellet comprising uniform concentrations of the microbe-based composition per pellet. Methods known in the art for producing feed pellets can be used to produce them, including pressurized milling. Preferably, the pelleting process is “cold” pelleting, or a process that does not use high heat or steam.

In one embodiment, particularly for use in aquatic applications, microbe strains are cultured for the purpose of producing an inactive microbe-based composition. The composition is prepared by cultivating the desired microorganism, inactivating the microbe by micro-fluidizing (or by any other method known in the art not to cause protein denaturation), pasteurizing and adding it to the food stuff in concentrated form. In one embodiment, inactivation occurs at pasteurization temperature (up to 65° to 70° C. for a time period sufficient to inactivate 100% of the yeast cells) and increasing pH value up to about 10.0. This induces partial hydrolysis of cells and allows for freeing of some nutritional components therein. Then, the composition is neutralized to a pH of about 7.0-7.5 and the various components of hydrolysis are mixed. The resulting microbe-based product can then be used for, for example, fish feed and treatment of fish farm water.

The microbe-based products of the subject invention are particularly advantageous compared to traditional products wherein cells have been separated from metabolites and nutrients present in the fermentation growth media.

Microbial Strains Grown in Accordance with the Subject Invention

The microorganisms grown according to the systems and methods of the subject invention can be, for example, bacteria, yeast and/or fungi. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In preferred embodiments, the microorganism is not genetically modified.

In certain preferred embodiments, the microorganism is any yeast known as a “killer yeast.” As used herein, “killer yeast” means a strain of yeast characterized by its secretion of toxic proteins or glycoproteins, to which the strain itself is immune. Such yeasts can include, but are not limited to, Wickerhamomyces (e.g., W. anomalus), Pichia (e.g., P. anomala, P. guilliermondii, P. occidentalis, P. kudriavzevii), Hansenula, Saccharomyces, Hanseniaspora, (e.g., H. uvarum), Debaryornyces (e.g., D. hansenii), Candida, Cryptococcus, Kluyveromyces, Torulopsis, Ustilago (e.g., U. maydis), Williopsis, Zygosaccharomyces (e.g., Z. bailii), and others.

In a specific embodiment, the subject invention utilizes phytase-producing killer yeasts. Even more specifically, the microbes of the subject invention include Wickerhamomyces anomalus (Pichia anomala).

These yeasts have a number of beneficial characteristics useful for the present invention, including their ability to produce advantageous metabolites. For example, W. anomalus is capable of exo-β-1,3-glucanase activity, making it capable of controlling or inhibiting the growth of a wide spectrum of pathogenic fungi. Additionally, if cultivated for 5-7 days, W. anomalus produces glycolipid biosurfactants that are capable of reducing surface/interfacial tension of water, as well as exhibiting antimicrobial and antifungal properties.

In addition to various by-products, these yeasts are capable of producing phytase and providing a number of proteins (containing up to 50% of dry cell biomass), lipids and carbon sources, as well as a full spectrum of minerals and vitamins (B1; B2; B3 (PP); B5; B7 (H); B6; E).

Other microbial strains including, for example, other microbial strains capable of accumulating significant amounts of, for example, enzymes (particularly phytase), acids, proteins, biosurfactants, minerals or vitamins that are useful in enhancing production in human health, agriculture, horticulture, landscaping, forestry, livestock rearing and aquaculture, can also be used in accordance with the subject invention. In some embodiments, other members of the Pichia and/or Wickerhamomyces clades are utilized, e.g., P. anomala, P. guilliermondii, P. occidentalis, and/or P. kudriavzevii.

Methods of Liberating Phosphates from Organic Matter

In certain embodiments, the subject invention provides environmentally-friendly, cost-efficient materials and methods for liberating phosphates from phytic acid containing-organic matter. In one embodiment, methods are also provided for enriching soil to increase crop growth and yields utilizing compositions of the subject invention. In another embodiment, methods are provided for feeding livestock and/or farmed fish utilizing compositions of the subject invention. In yet another embodiment, methods are provided for enhancing the health of a human subject utilizing compositions of the subject invention.

The methods allow for the recycling of organic waste material, as well as the release of vitamins, minerals and importantly, phosphorus, that remain therein. Furthermore, the compositions and methods utilize components that are biodegradable and toxicologically safe. Thus, the present invention can be used for enhancing production in agriculture, forestry, and animal husbandry, as well as for enhancing human health, as a “green” treatment.

In certain embodiments, the subject methods are used for liberating phosphates from phytic acid present in organic matter, wherein the methods comprise applying an effective amount of a microbe-based composition of the subject invention to the organic matter. Further components can also be applied, such as, for example, water or other nutrients (e.g., nutrients and/or prebiotics). The microbes can be either live (viable) or inactive at the time of application.

In the case of live microorganisms, the microorganisms can grow in situ at the site of application and produce any active compounds or growth by-products onsite. Consequently, a high concentration of microorganisms and beneficial growth by-products can be achieved easily and continuously at a treatment site.

To this end, the methods can comprise adding materials to enhance microbial growth during application (e.g., adding nutrients to promote microbial growth).

In one embodiment, the methods further comprise a step of cultivating the microbe-based composition prior to application. Preferably, all or part of the microbe-based composition is cultivated at or near the site of application, for example, less than 300 miles from the site.

As used herein, “applying” a composition or product to a target or site, or “treating” a target or site refers to contacting a composition or product with a target or site such that the composition or product can have an effect thereon. The effect can be due to, for example, microbial growth and/or the action of a metabolite, enzyme, biosurfactant or other growth by-product. Advantageously, when the composition of the subject invention is contacted with organic matter according to the subject methods, the phytase in the composition can catalyze the hydrolysis of the phytate or phytic acid in the organic matter, causing a release of phosphorus byproducts that are absorbable by plants, humans and animals, e.g., in the form of inorganic phosphates.

In one embodiment, application of the subject microbe-based composition comprises pouring, spraying or sprinkling the composition onto organic matter and then, optionally, mixing the composition with the organic matter using any standard mixing device or technique known in the art.

In one embodiment, the subject microbe-based composition can be applied to organic matter that has been collected from its source and mixed at another location. The treated organic matter can then be transported to a desired application site, such as, for example, a crop field and used as, for example, a biofertilizer.

In certain embodiments, the organic matter that is treated according to the subject methods is organic waste matter, such as post-harvest crop residue, which can include, for example, leftover corn stalks, corn stover, corn cobs, wheat straw, soybean straw, rice hulls, and other plant stems, leaves, roots and parts. Other types of organic waste are also envisioned, including, for example, plant-based compost, manure, leftovers from corn, cellulosic or biomass ethanol production (e.g., distiller's grains, lignin and brewers' spent grain), saw dust, used coffee grounds, and yard waste (e.g., tree, hedge and lawn clippings).

In certain embodiments, the organic matter that is treated according to the subject methods is plant matter containing phytate or phytic acid. For example, the organic plant matter can be seeds (e.g., linseed, flax seed, rape seed, soybeans, and sunflower seeds), potatoes, grains (e.g., wheat, rice, bran, barley, corn, rye, and oats), legumes (e.g., pinto beans, kidney beans, navy beans and peanuts) or nuts (e.g., almonds, Brazil nuts, hazelnuts and walnuts).

In some embodiments, the speed at which phosphate release occurs can be enhanced by chopping, crushing or otherwise reducing the size of any individual pieces of the organic matter prior to applying the microbe-based composition.

In one embodiment, the organic matter is crop residue that is leftover on a post-harvest crop field. As crop residue decomposes, nutrients that are necessary for plant growth are released into the soil. The subject invention can be used to convert unavailable forms of phosphorus that are released by this decomposition process into plant-absorbable forms. For example, the composition can be applied directly onto crop residue that is left behind on a field. The crop residue and composition can be left on the surface of the soil, or they can be tilled into the soil.

In one embodiment of the subject methods, the organic matter is soil. The microbe-based composition of the subject invention can be applied directly to the soil, or, organic matter that has been pre-treated with the microbe-based composition can be applied to the soil. Optionally the composition and/or the pre-treated organic matter can be mixed into the soil, for example, by tilling, or the composition can be allowed to percolate into the soil without mixing.

In certain embodiments, the composition is applied to the soil surface without mechanical incorporation. The beneficial effect of the soil application can be activated by rainfall, sprinkler, flood, or drip irrigation, and subsequently delivered to, for example, the roots of plants.

In one embodiment, the method can enhance plant health, growth and/or yields by enhancing root health and growth. More specifically, in one embodiment, the methods can be used to improve the properties of the rhizosphere in which a plant's roots are growing, for example, the nutrient and/or moisture retention properties.

Additionally, in one embodiment, the method can be used to inoculate a rhizosphere with one or more beneficial microorganisms. For example, in preferred embodiments, the microbes of the subject composition can colonize the rhizosphere and provide multiple benefits to the plant whose roots are growing therein, including protection and nourishment.

Plants and/or their environments can be treated at any point during the process of cultivating the plant. For example, the composition can be applied to the soil prior to, concurrently with, or after the time when seeds are planted therein. It can also be applied at any point thereafter during the development and growth of the plant, including when the plant is flowering, fruiting, and during and/or after abscission of leaves.

In one embodiment, the method can be used in a large scale forestry and/or agricultural setting. The method can comprise administering the composition into a tank connected to an irrigation system used for supplying water, fertilizers or other liquid compositions to a crop, forest, pasture, orchard or field. Thus, the plant and/or soil surrounding the plant can be treated with the soil treatment composition via, for example, soil injection, soil drenching, or using a center pivot irrigation system, or with a spray over the seed furrow, or with sprinklers or drip irrigators. Advantageously, the method is suitable for treating hundreds of acres of crops, forest, pastures, orchards or fields at one time.

In one embodiment, the method can be used in a smaller scale setting, such as in a home garden, in municipal landscaping, or in a greenhouse. In such cases, the method can comprise application using irrigation systems, a handheld lawn and garden sprayer, and/or a standard handheld watering can.

Advantageously the subject methods can: promote germination of seeds; increase survival of seedlings and young trees in reforestation and landscaping; increase crop yields; and enhance the quality of produce and plant products grown in this enriched soil due to, for example, the presence of beneficial microbial metabolites such as phytase, amino acids, proteins, vitamins and microelements. Additionally, the increased growth and health of plants is an important means of reducing atmospheric carbon levels, as the increase in plant biomass (as well as the associated soil microbial biomass) sequesters increased levels of carbon. Furthermore, the presence of phytase in a plant's growing environment (e.g., soil) allows for the treatment and/or prevention of phosphorus deficiency in plants.

In certain embodiments, due to the presence of advantageous biochemical-producing microorganisms in the subject microbe-based compositions, the subject methods can also help with preventing harmful organisms from harboring in organic waste matter. For example, manure and decomposing crop residue can be attractive for certain pests, fungi and bacteria that might be harmful to plants that are grown with the manure or residue. Killer yeasts, such as Wickerhamomyces anomalus, are capable of producing metabolites that are useful for controlling many of these unwanted pests.

In one embodiment, methods of enhancing production in animal husbandry (e.g., livestock rearing or aquaculture) are provided, wherein the microbe-based composition is formulated with, or applied to, an animal's feed or drinking water as a dietary supplement and/or a digestive aide. As a food supplement and/or digestive aide, the microbe-based products can provide, among other benefits, sources of amino acids, proteins, vitamins and microelements, as well as aiding in phosphorus absorption in an animal's digestive tract due to the presence of phytase.

In the case of livestock, the microbe-based composition can be introduced into feeding troughs alongside traditional livestock feed, and the animals are then allowed to ingest the composition. In one embodiment, the composition can be mixed in with feed components and formulated into uniform, homogenized pellets.

In the case of farmed fish, the composition can be applied to a fish's environment, such as fish farm water, in the form of, for example, a liquid solution, or as dry powder, meal, or feed flakes or pellets.

In one embodiment, methods of enhancing human health are provided, wherein the microbe-based composition is administered to a human subject as a dietary supplement and/or a digestive aide. In preferred embodiments, the composition is administered orally.

As a dietary supplement and/or digestive aide, the compositions and methods can provide, among other benefits, sources of amino acids, proteins, vitamins and microelements, as well as aiding in phosphorus absorption in the subject's digestive tract due to the presence of phytase. Additionally, the compositions can help prevent phytic acid from impairing the absorption of minerals, such as, for example, magnesium, calcium, zinc, iron, manganese, copper and/or molybdenum, by the subject's digestive tract, thereby preventing deficiencies of these minerals.

Advantageously, when a human or animal ingests the composition, the presence of phytase in the composition allows for enhanced growth and health by, for example, increasing absorption of phosphorus from food sources that may naturally contain phosphates and/or phytic acid; reducing the amount of inorganic phosphate needed to supplement food; and helping treat and/or prevent phosphorus deficiency and/or other mineral deficiencies related to ingestion of phytic acid.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—Yeast Fermentation for Phytase Production

A yeast fermentation product resulting from cultivation of yeasts, for example, Wickerhamomyces anomalus (Pichia anomala), can be useful for liberating phosphorus from phytic acid- or phytate-containing organic matter.

The fermentation broth after 48-72 hours of cultivating W. anomalus at 25-30° C. can contain the yeast cell suspension and up to, for example, 9,000 u/mL of phytase, as well as other yeast growth by-products and cellular components. The cells can be separated from the liquid media, or kept therein.

The yeast fermentation product (liquid media with phytase and optionally live or inactive yeast cells) can be applied directly to organic matter, soil, plant roots, water, animal feed and/or dietary supplements. It can be used directly upon harvesting, or stored and transported to a desired treatment location. Advantageously, the subject methods can be useful for, e.g., phosphorus conservation and for resolving the phosphorus depletion in the environment.

### Example 2—Calculating Phytase Production Using Wickerhamomyces anomalus

Wickerhamomyces anomalus was grown in a fermentation reactor at a pH adjusted to 5.0 for 72 hours with the following growth medium: molasses (2.0-5.0%, or 3.0-4.0%), urea (0.1-1.3%, or 0.15-0.2%), peptone (2.0-3.0%, or 2.2-2.5%). Typical microbial production of phytase using other microorganisms and/or using other growth medium formulations result in lower concentrations of phytase, about 300 to 500 u/ml.

Phytic acid was taken from sesame seeds by breaking/crushing the seeds. The seed parts (5 g) were then added to 10 mL of water and mixed. The media and cells were tested for the presence of phytase. 0.2 M glycine buffer was used to adjust pH to 4 and pH of phytic acid was also adjusted to 4.

After 3 days, phytase activity was measured by preparing 6 different standards:
0) 550 microliters of water
1) 10 microliters of 50 mM potassium phosphate and 540 microliters of water
2) 20 microliters of 50 mM potassium phosphate and 530 microliters of water
3) 30 microliters of 50 mM potassium phosphate and 520 microliters of water
4) 40 microliters of 50 mM potassium phosphate and 510 microliters of water
5) 50 microliters of 50 mM potassium phosphate and 500 microliters of water

Fifteen phytic acid blanks were prepared using 500 μL phytic acid, 25 μl 0.2 M glycine buffer and 25 μl water.

Fifteen phytase sample blanks were prepared using 25 μl phytase, 25 μl 0.2 M glycine buffer and 500 μl water.

Fifteen phytase samples were prepared using 500 μL phytic acid, 25 μl 0.2 M glycine buffer and 25 μl phytase.

A solution of 4 ml of aceton-ammonium molybdate-H₂SO₄ (50 ml-25 ml-25 ml) was added to each of the 6 standards.

Each of the samples and blanks were transferred to a water bath for 30 minutes at 37° C. After 30 minutes, 4 ml of the aceton-ammonium molybdate-H₂SO₄ solution was added quickly to each of the samples and blanks to precipitate the phosphate. Then they were centrifuged and 300 μl of supernatant was dissolved in 600 μl of water.

Next, 500 μl of all samples, blanks and standards were uploaded into Varioscan equipment well plates and absorbance was measured at 400 nm.

Standard values were measured 3 time and their averages were taken. Standard 0 value was subtracted from each of values of standards 1-5 to achieve a number correlated to the absorbance of a particular concentration of phosphate. Average absorbance for each of the standards is reported in Table 1. Average absorbance of samples and blanks is reported in Table 2.

TABLE 1
Average absorbance (OD) and phosphate
(mM) levels measured for standards 0-5.

| Standard | Absorbance (OD) | Phosphate (mM) |
|---|---|---|
| 0 | 0 | 0 |
| 1 | 0.0456 | 0.9091 |
| 2 | 0.1426 | 1.8182 |
| 3 | 0.1696 | 2.7273 |
| 4 | 0.2566 | 3.6364 |
| 5 | 0.2726 | 4.5455 |

TABLE 2
Average absorbance (OD) of samples and blanks.

| Sample/Blank | Absorbance (OD) |
|---|---|
| Phytic Acid Blank | 0.26 |
| Phytase Sample Blank | 0.047 |
| Phytase Sample | 0.319 |

To determine the millimoles (mM) of phosphate released, average absorbance of the phytase sample blanks were added to the average absorbance of phytic acid blanks and subtracted from the average absorbance of the phytase sample. The resulting number was 0.012 OD. The value of the phosphate released was 0.198 mM.

$$units \text{/} ml of enzyme activity = \frac{\begin{matrix}( mM of phosphate ) \times \\ ( dilution factor )\end{matrix}}{( time ) \times ( amount of enzyme added )} \qquad ( 1 )$$

Finally, according to Equation 1 above, phytase concentration in the reactor was calculated to be 792 units/ml.

1. A composition for liberating phosphates from phytic acid present in organic matter, the composition comprising a Wickerhamomyces anomalus yeast and/or one or more microbial growth by-products of said Wickerhamomyces anomalus, and a carrier.

2. The composition of claim 1, wherein the growth by-product is phytase.

3. The composition of claim 1, comprising fermentation broth resulting from cultivation of the yeast.

4. (canceled)

5. The composition of claim 1, comprising one or more biosurfactants.

6. The composition of claim 5, comprising one or more biosurfactants selected from glycolipids lipopeptides, flavolipids, phospholipids, fatty acid ester compounds, lipoproteins, lipopolysaccharide-protein complexes, and polysaccharide-protein-fatty acid complexes.

7-8. (canceled)

9. A method for liberating phosphorus from phytic acid in organic matter, the method comprising applying to the organic matter a composition of claim 1.

10. (canceled)

11. The method of claim 9, wherein the organic matter is selected from crop residue, manure, compost, yard waste, coffee grounds, sawdust, distiller's grains, lignin and brewers' spent grain.

12. The method of claim 11, wherein the organic waste-matter is crop residue that is spread over the surface of a crop field.

13-14. (canceled)

15. The method of claim 9, wherein the organic matter is soil.

16. A method for enriching soil, wherein a composition of claim 1 is applied to the soil thereby liberating phosphates from phytic acid present in the soil.

17. The method of claim 16, wherein organic waste that has been pre-treated with the composition is applied to the soil.

18. The method of claim 16, used to enhance production in agriculture or forestry, wherein a plant is grown or will be grown in the enriched soil.

19. (canceled)

20. The method of claim 18, used to treat and/or prevent phosphorus deficiency in the plant.

21. A method of enhancing production in animal husbandry, wherein the method comprises applying an effective amount of a composition comprising a Wickerhamomyces anomalus yeast and/or one or more microbial growth by-products of said Wickerhamomyces anomalus, and, optionally a carrier, to, or formulating the composition with, an animal's drinking water and/or feed as a dietary supplement and/or digestive aide.

22. The method of claim 21, wherein the animal husbandry is livestock rearing.

23. The method of claim 21, wherein the animal husbandry is aquaculture.

24. The method of claim 21, used to treat and/or prevent phosphorus deficiency in the animal.

25. A method of enhancing human health, wherein the method comprises administering an effective amount of a composition of claim 1 to a human subject as a dietary supplement and/or digestive aide.

26. (canceled)

27. The method of claim 25, used to treat and/or prevent phosphorus deficiency in the subject.

28. The method of claim 25, used to prevent phytic acid from impairing absorption of minerals by the subject's digestive tract.
